(19) United States
(12) Patent Application Publication
(10) Pub. No.: US 2009/0258085 A1
(43) Pub. Date: Oct. 15, 2009

(54) Emulsions and methods of their production

(75) Inventors: Uri Meir Bach, Efrat (IL); Zeev Maor, Dead Sea (IL)

Correspondence Address: THE NATH LAW GROUP, 112 South West Street, Alexandria, VA, 22314, US

(73) Assignee: AHAVA- DEAD SEA LABORATORIES LTD., Mobile Post Dead Sea (IL)

(21) Appl. No.: 12/382,467

(22) Filed: Mar. 17, 2009

Related U.S. Application Data
(60) Provisional application No. 61/064,617, filed on Mar. 17, 2008.

(51) Int. Cl. A61K 8/06; A61K 33/00; A61P 17/00

(52) U.S. Cl. 424/600

(57) ABSTRACT

An emulsion including Dead Sea water is provided, as well as methods for making and using such an emulsion. The emulsion can also include a structured surfactant.

### FIELD OF THE INVENTION

This invention relates to emulsions and methods for their production.

### BACKGROUND OF THE INVENTION

It is often desirable in the cosmetic/personal care product industry to provide one or more active ingredients as an emulsion, which as known in the art typically consist of a suspension of the active ingredient in the form of, e.g., oil, in an aqueous solution. In some cases a surfactant is introduced.

Recently, structured surfactants have been proposed as an alternative to the common surfactants, particularly where the suspension comprises large particles. Structured surfactants comprise of a surfactant mesophase or solid phase, usually a lamellar or G-phase, alone or more usually interspersed with an aqueous phase, and are characterized as being fluidic and non-Newtonian compositions, having the capacity to physically stabilize emulsions by virtue of the presence of the surfactant mesophase or solid phase.

### SUMMARY OF THE INVENTION

The inventors of the present invention have now demonstrated that incorporation of Dead Sea water (DSW) in emulsions, particularly those for human and animal use, endows the emulsions not only with one or more of the advantages associated with products of the Dead Sea but surprisingly also with an unexpected increase in stability and further still increased viscosity, without increasing their stickiness (the unpleasant organoleptic feeling associated with emulsions). Emulsions of the invention have demonstrated an extensive stability over long periods of time, in excess of two years, even when high salt concentrations, typically known to cause separation of emulsions, have been used, as further demonstrated below.

Thus, in one aspect of the present invention, there is provided an emulsion comprising a high concentration salt solution, e.g., wherein said salt solution constitutes at least 10% of the total volume of the emulsion. In some embodiments, the salt solution is DSW. Such emulsions of the invention are considered stable emulsions, namely they do not each separate into an aqueous phase and oil phase over time. The emulsions of the present invention have demonstrated such stability over periods exceeding 2 years.

In some emulsions of the invention, the high concentration salt solution is DSW and the emulsion further comprises at least one structured surfactant. In still further embodiments, the emulsion of the invention comprises water, at least one oil, Dead Sea water (DSW), at least one structured surfactant, and optionally at least one additive.

The water may be water alone or a salt solution, e.g., having any concentration of salts (electrolytes), or a homogeneous water solution comprising other water soluble additives such natural extracts, colorants and dyes, perfumes, active ingredients, stabilizers, soluble polymers (e.g., cationic polymers) and other water-soluble materials. The water or solution may be added to the emulsion purified, e.g., distilled (once distilled, doubly distilled, triply distilled, etc), or as a solution or may originate, e.g., from the water of the DSW employed. In some embodiments, the water of the emulsion is a combination of water of the Dead Sea and water or a salt solution prepared independently.

The at least one oil comprises a substantially water-insoluble material, which may be in a liquid or solid state, in some embodiments in the form of a water-insoluble oil selected from at least one vegetable oil, at least one essential oil, at least one botanical oil, at least one mineral oil, at least one animal oil and at least one lipid.

In some embodiments, the oil is at least one vegetable oil or a mixture of vegetable oils. The at least one vegetable oil may be selected from almond, coconut, corn, castor bean, evening primrose, groundnut, meadow foam, apricot kernel, peach kernel, peanut, sunflower, safflower, soybean, avocado, rapeseed, jojoba walnut, and olive oil.

In some embodiments, the oil is at least one essential oil, at least one botanical oil at least one lipid and mixtures thereof. “Botanical oils” are oils obtained from any one variety of plant. “Essential oils” are those that in general give to the plants their characteristic odors, flavors, or other such properties. “Lipids” as referred to herein include a fatty acid, a glycerol-derived lipid (including the fats and oils and the phospholipids), a sphingosine-derived lipid (including the ceramides, cerebrosides, gangliosides, and sphingomyelins), a steroid and derivatives thereof, a terpenes and derivatives thereof, an aromatic compound, a long-chain alcohol and a wax. The term also refers to a lipoprotein (lipids conjugated with proteins or carbohydrates) and to vitamins such as fat-soluble vitamins.

In some embodiments, the essential oil is selected from cinnamon oil, cedar oil, clove oil, geranium oil, lemongrass oil, mint oil, sesame oil, thyme oil, turmeric oil, wintergreen oil, rosemary, anise oil, cardamom oil, chamomile oil, coriander oil, cumin oil, dill oil, mint oil, parsley oil, basil oil, camphor oil, citronella oil, eucalyptus oil, fennel oil, ginger oil, grapefruit oil, lemon oil, mandarin oil, orange oil, pine needle oil, pepper oil, rose oil, sweet orange oil, tangerine oil, tea tree oil, tea seed oil, caraway oil, garlic oil, peppermint oil, onion oil, citronella oil, lavender oil, clove pine oil, eucalyptus oil and spearmint oil.

In some embodiments, the emulsion comprises at least one mineral oil, e.g., low molecular weight petroleum oil, a fatty glyceride or an ester (e.g., lauryl acetate), a terpene oil (e.g., limonene) or silicone oil.

The oil may be in the form of a mixture of one or more such oils, which may be of the same group of oils (e.g., two or more vegetable oils) or each from a different group of oils (e.g., at least one vegetable oil and at least one mineral oil).

In other embodiments, the oil is animal oil or a mixture thereof. In further embodiments, the oil is a mixture of vegetable and animal oils. Yet in further embodiments, the oil is generally selected amongst non-plant origins.

In some embodiments, the emulsion of the invention comprises at least about 3% w/v oil. In some other embodiments, the oil constitutes at most about 30% w/v of the emulation. In further embodiments, the oil constitutes between about 3 and 30% w/v of the emulsion.

“Dead Sea water” (herein abbreviated DSW) refers in most general terms to an aqueous solution which is obtained from the Dead Sea (Israel) or an aqueous solution which simulates such a natural solution, namely having at least one parameter substantially identical to that measured for the natural DSW, said parameter being at least one of salt content, salt concentration, concentration of a particular cation or anion, ratio of divalent cations to monovalent cations, TDS (Total Dissolved Salt, w/v), soluble natural substances, and other parameters known to define or characterize natural DSW.

Typically, the DSW employed in the emulsion of the present invention is natural DSW obtained from the Dead Sea or a simulant thereof having a TDS value of at least 17% and/or at least 88,000 mg/L of divalent cations (e.g., calcium and/or magnesium). In some embodiments, the ratio of divalent cations to monovalent cations (e.g., sodium and/or potassium) in the DSW is typically 20:1; 30:1; 35:1; 40:1 or 45:1 (or intermediate or greater values), respectively.

In further embodiments, the DSW is natural DSW obtained from the Dead Sea region.

In some further embodiments, the DSW employed is natural DSW obtained from the Dead Sea and comprising at least one of the following ions:

| | | |
|---|---|---|
| Calcium (Ca⁺²) | 34,000-40,000 | mg/L |
| Chloride (Cl⁻) | 320,000-370,000 | mg/L |
| Magnesium (Mg⁺²) | 90,000-95,000 | mg/L |
| Potassium (K⁺) | 1,300-2,200 | mg/L |
| Sodium (Na⁺) | 1,500-2,800 | mg/L |
| Bromide (Br⁻) | 11,000-15,000 | mg/L |

In some further embodiments, the DSW is natural DSW which has undergone pre-treatment. In further embodiments, the DSW is concentrated by allowing water to evaporate, e.g., through solar evaporation, thereafter reconstituted to afford a solution (such as the commercially available Maris Sal, AHAVA, Israel) having an overall salt concentration (constituting the original salt composition) of 15, 20, 25, 30, 35, 40 or 45% or intermediate or greater concentrations.

In some further embodiments, the emulsion of the invention comprises at least about 10% w/v DSW. In some other embodiments, the emulsion comprises at most about 30% w/v of DSW. In further embodiments, the emulsion comprises between about 10 and 30% w/v DSW.

As exemplified herein, the invention provides emulsions having 10, 15, 20, 25 or 30% DSW, and other intermediary concentrations. Surprisingly, concentrations of DSW in the range of about between 10 and 20% w/v are sufficient to reduce emulsion particle size in the short term with the benefit of higher concentrations of DSW becomes apparent after longer term storage. Beneficial effects of higher concentrations of DSW can be apparent in emulsion stability and/or activity on skin. The beneficial effects and increased stability of emulsions comprising DSW are apparent when compared to other emulsions.

As used herein, the term “structured surfactant” refers to a pourable, fluid, non-Newtonian (i.e., flow properties not defined by a single constant value of viscosity) surfactant composition having the capacity to physically stabilize an emulsion by virtue of the presence of a surfactant mesophase or solid phase, which may be interspersed with a solvent phase; the solvent phase being water or an aqueous electrolyte phase and the surfactant phase comprising packed spherulites dispersed in the solvent phase or in a thin mobile lamellar phase or a bicontinuous reticular interspersion of aqueous and lamellar phases.

In some embodiments, the structured surfactant is a combination of at least one surfactant selected from at least one anionic surfactant, at least one nonionic surfactant, at least one amphoteric surfactant, at least one zwitterionic surfactant and at least one cationic surfactant with at least one agent selected from water, at least one anionic surfactant, at least one electrolyte, and at least one alkanolamide, wherein in some embodiments the combination of the at least one surfactant, as defined, and said at least one agent possesses non-Newtonian shear thinning properties and a stable viscosity under at least one freeze/thaw cycle.

In some further embodiments, the structured surfactant is a combination of at least one of sodium trideceth sulfate, sodium lauro amphoacetate, and cocamide monoethanolamine (MEA); said combination being commercially available (SLB 365, Rhodia; Cranbury N.J.; USA).

In some embodiments, the structured surfactant, e.g., SLB 365 may also comprise at least one preservative such as DMDM hydantoin.

Exemplary structured surfactants are disclosed, for example, in international patent publication no. WO 2003/055456 or in US application no. US2003180246, being fully incorporated herein by reference. Additional structured surfactant compositions and methods of their production are disclosed in the following patents and patent applications: EP586275; EP586275; U.S. Pat. No. 5,556,628; WO03055456; US20030180246; EP1458337; WO03055455; EP1465584; US20030190302; WO2005009385; EP1670426; US20050020468; WO2005055937; EP1692254; US20050124526; EP1747260; US20050233935; WO2005103221; WO2006023548; US20060040837; EP1786893; WO2006127394; US20060270584 and US20060135627, all being incorporated herein by reference.

In some embodiments, the structured surfactant is characterized by at least one non-Newtonian shear thinning property and a stable viscosity following a shelf life acceleration stability model of product incubation in one or more freeze/thaw cycles, e.g., 45°-20°-4° C. cycles.

In further embodiments, the emulsion of the invention comprises at least about 20% w/v of said at least one structured surfactant. In further embodiments, the emulsion comprises at most about 40% w/v of at least one structured surfactant. In some further embodiments, emulsions of the invention comprise between about 20% to 40% of said at least one structured surfactant and between about 3% to 30% of said at least one oil.

The emulsions of the invention may optionally further comprise at least one additive in an amount not exceeding 20% w/v. Such additives are not necessary for the stability of the emulsion and may be introduced to induce or provide additional advantage or characteristics required for one or more additional applications. The at least one additive may be selected, in a non-limiting fashion from a co-surfactant, a dye, a colorant, a perfume, an optical brightener, a stabilizer and a co-solvent (such as ethanol or isopropyl alcohol, ethylene glycol, isopropylene glycol, glycerol or water miscible glycol ethers such as ethylene glycol monomethyl ether, diethylene glycol monomethyl ether or polyethylene glycol).

In some further embodiments, the at least one additive is at lease one cation of a polymeric source. Without wishing to be bound by theory, cationic polymers assist in the conditioning of emulsions. At certain concentrations cations of a polymeric source may also provide the emulsion with increased viscosity. The increased viscosity is usually accompanied by the stickiness feeling commonly associated with such emulsions. In order to reduce/avoid stickiness, in some embodiments of the invention, the concentration of cationic polymers in the emulsion is reduced or minimized, e.g., substantially to zero.

In further embodiments, the at least one additive is at lease one cation of a non-polymeric source (e.g., DSW). Without wishing to be bound by theory, cations of a non-polymeric source increase the viscosity of the emulsion and significantly reduce the stickiness associated, and additionally result in an improved tactile quality as evaluated subjectively by consumers of products such as shampoos, hair mousse and/or gel and various creams and/or lotions.

In some embodiments, the emulsions of the invention further comprise salts and other material (e.g., mud and minerals) obtained from the Dead Sea. In some other embodiments, the emulsions comprise at least one dye, at least one colorant, and/or at least one perfume.

The emulsions of the invention may be used in any one cosmetic, pharmaceutical or nutraceutical industry as a system for suspending at least one solid, liquid or a gaseous particle. Oil or water soluble cosmetic or topical pharmaceutical ingredients which may be dissolved in the emulsion include, in a non-limiting fashion, at least one agent selected from an antiseptic, an antihistamine, a styptic, an antidandruff agent (e.g., zinc omadine and selenium disulphide), a protein, an emollient (e.g., lanolin, isopropyl myristate, glyceryl isosterate and propylene glycol distearate), a wax, an exfoliant (e.g., talc, clay, polymer beads, sawdust, silica, seeds, ground nutshells and calcium phosphate), a pearliser (e.g., mica, glycerol and ethylene glycol distearate), a glitter additive and a sunscreen material (e.g., titanium dioxide). The emulsion may alternatively or additionally be used to suspend micro- or nano-encapsulated ingredients (active or inert).

The emulsions of the invention may also comprise at least one active ingredient, free or encapsulated, for the topical treatment or prevention of a skin disease or disorder. Non-limiting examples of such skin disease or disorder include dermatological inflammation; different acne types such as acne vulgaris, cystic acne, acne rosacea, acne keloidalis nuchae, acne conglobata, acne cosmetica, acne fulminans, acne medicamentosa, baby acne and Chloracne; various kinds of dermatitis; different infections such as bacterial skin infections, fungal and yeast skin infections, viral skin infections, parasitic skin infections; pruritis; cellulites; acute lymphangitis; lymphadenitis; erysipelas; cutaneous abscesses; necrotizing subcutaneous infections; scalded skin syndrome; folliculitis; furuncles; hidradenitis suppurativa; carbuncles; paronychial infections; rashes; erythrasma; impetigo; warts; molluscum contagiosum; trauma or injury to the skin (wounds); post-operative or post-surgical skin conditions; pediculosis; creeping eruption; eczemas; different types of psoriasis; pityriasis rosea; lichen planus; pityriasis rubra pilaris; edematous; erythema multiforme; erythema nodosum; grannuloma annulare; epidermal necrolysis; sunburn; photosensitivity; pemphigus; bullous pemphigoid; dermatitis herpetiformis; keratosis pilaris; callouses; corns; ichthyosis; skin ulcers; ischemic necrosis; miliaria; hyperhidrosis; moles; poison ivy; poison oak; contact dermatitis; atopic dermatitis; rosacea; purpura; moniliasis; candidiasis; baldness; alopecia; Behcet's syndrome; cholesteatoma; Dercum disease; ectodermal dysplasia; gustatory sweating; nail patella syndrome; lupus; hives; hair loss; Hailey-Hailey disease; chemical or thermal skin burns; scleroderma; aging skin; wrinkles; sun spots; necrotizing fasciitis; necrotizing myositis; gangrene; scarring; athlete's foot; ringworm and vitiligo.

The active ingredient may be in the form of a drug molecule (e.g., minoxidil) or a plant (e.g., herbal) extract.

The emulsions of the invention may also comprise at least one active ingredient for skin protection, e.g., anti UVA or UVB agents, sunscreen agents or sun-tanning agents.

The emulsion of the invention may additionally be formulated for human and/or animal use as skin care products for a variety of applications. In some embodiments, the emulsions of the invention are formulated as personal skin care products selected from a cleansing product and a moisturizing product. In some embodiments the cleansing product is selected from a shampoo, a liquid soap and a bath/shower gel. In some further embodiments the moisturizing product is selected from a cream, a lotion, a gel-cream, a conditioner and a mask.

Generally, the emulsions of the invention and formulations comprising same are suitable and safe for topical application onto the skin (any part of the animal skin including whole skin, hair and nails) of a subject (human or non-human) for any period of time which is effective to achieve, induce or prevent a certain end result. In some embodiments, the emulsions of the invention are used in the treatment of at least one disease or disorder associated with the skin, as detailed above. In other embodiments, the emulsions are used in a method for preventing at least one symptom associated with such a skin condition. In some embodiments, the emulsions of the invention are used for protecting the skin of a subject from UV-induced disease or disorder. In some embodiments, the UV-induced disease or disorder is apoptosis or inflammation.

In some further embodiments, the subject is suffering, or has predisposition to suffer, or is one which may be exposed to conditions which increase the chances of suffering from a disease or disorder of the skin, which is optionally (may or may not be) related to one or more of age, sex, skin color, skin wounds, exposure to the sun, UV radiation, inflammation, a pre-existence of a disease not associated with the skin, etc.

In some embodiments, the disease or disorder of the skin is related to sun exposure.

The term “topical” as used herein refers to the application of an emulsion according to the invention directly onto at least a portion of a subject's skin so as to achieve a desired effect, e.g., cosmetic or therapeutic effect, at the site of application. In some embodiments, the desired effect is achieved at the site of application without inducing one or more systemic effects. In other embodiments, the emulsion of the invention induces at least a partial systemic effect which contributes to the induction of at least one desired effect.

As used herein, “treatment” or “prevention” refers to the topical administration of an effective amount of an emulsion of the present invention effective to ameliorate undesired symptoms associated with a skin disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The “effective amount”, whether therapeutically or cosmetically effective amount for purposes herein is determined by such considerations as may be known in the art. The amount or any ratio between two or more of the emulsion's components must be effective to achieve one or more of the above desired therapeutic or cosmetic effects, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile, a variety of pharmacological parameters such as half life on the skin, on undesired side effects, if any, on factors such as age and gender, etc.

Emulsions of the invention, as such or as formulated into a topical formulation, may be applied onto the skin by any one method known for application of a standard cream. The application may be for a short period of time, namely the emulsion in a suitable form (as disclosed herein) is applied topically and then removed within a few minutes to 30 minutes. Alternatively, the emulsion may be applied and allowed to remain in contact with the skin over longer periods of time. In some embodiments, the emulation is allowed to remain on the skin overnight. In order to achieve long term effective contact with the skin, the emulsions of the invention may be absorbed or loaded onto a carrier which retains its form; such carrier may be a patch, a dressing or a bandage in a form providing sufficient contact with the skin.

For ease of use by the end user, the emulsions of the invention may be formed into a kit or a commercial package and provided along with instructions for use. The emulsions comprised in the kit or in the commercial package may be in a quantity and composition suitable for a short term or long term application, for a generic or specific purpose.

In a further aspect of the present invention, there is provided a method of preparing an emulsion according to the present invention, said method comprising combining (e.g., mixing or admixing) a high concentration salt solution such as DSW with a premade emulsion of at least one oil, at least one structured surfactant and water.

In some embodiments, the premade emulsion is obtained by mixing (e.g., admixing) at least one structured surfactant into water to form an aqueous matrix, thereafter mixing (e.g., admixing) into said aqueous matrix at least one oil.

In further embodiments, the emulsions of the invention are prepared by a method comprising:

(a) adding water into a mixing container;

(b) adding at least one structured surfactant to the mixing container (e.g., while mixing, in some embodiments, at a low speed of at least 60 rpm), to form an aqueous matrix;

(c) introducing at least one oil into the aqueous matrix (e.g., while mixing, in some embodiments, at a high speed of at least 300 rpm for a brief period of time), to thereby produce an initial emulsion; and

(d) optionally reducing the mixing speed and optionally continuing the mixing at the low speed of at least 60 rpm for a long period prior to adding an amount of DSW (e.g., while mixing, in some embodiments, at a low speed of at least 60 rpm for an additional period) to produce an emulsion according to the invention.

In some embodiments, in the method of the invention the at least one structured surfactant is characterized by non-Newtonian shear thinning properties and a stable viscosity under at least one freeze-thaw cycle.

In another of its aspects, the invention provides a method of producing a non-sticky emulsion, said method comprising

(a) combining water, at least one structured surfactant and at least one oil to produce an initial emulsion; and

(b) adding a non-polymeric source of cations, e.g., DSW, to thereby increase the viscosity of the emulsion without imparting stickiness to the emulsion.

The invention further provides in additional aspects skin care products comprising at least one emulsion of the present invention.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

The amount or concentration of each of the ingredients of any one emulsion of the invention may vary. It should be understood that any specific concentration of ingredients provided herein should be taken to mean an approximate concentration. For example, the expression “the emulsion comprises between about 20% to 40% of at least one structured surfactant” refers to a weight per volume (w/v) concentration which may from slightly below 20% to slightly above 40% or within the indicated range. For example, the range 20 to 40% would mean 19.5, 20.0, 20.1, 20.2, 20.3, 20.4, 20.5, 20.6, 20.7, 20.8, 20.9, 21.0, 21.1, 21.2, 21.3, 21.4, 21.5 and so on to 39.0, 39.1, 39.2, 39.3, 39.4, 39.5, 39.6, 39.7, 39.8, 39.9, 40.0, 40.1, 40.2, 49.3, 40.4, and 40.5%. Any equivalent amounts are within the scope of the present invention.

### BRIEF DESCRIPTION OF THE DRAWINGS

In order to understand the invention and how it may be carried out in practice, embodiments will now be described, by way of non-limiting examples only, with reference to the accompanying figures. In the figures, identical and similar structures, elements or parts thereof that appear in more than one figure are generally labeled with the same or similar references. Dimensions of components and features shown in the figures are chosen primarily for convenience and clarity of presentation and are not necessarily to scale. The attached figures are:

FIG. 1 demonstrates changes in average emulsion particle size as a function of DSW or salt (brine) concentrations over a period of 30 days.

FIG. 2 is a photograph of samples of different exemplary emulsions prepared using DSW or brine, illustrating improved stability.

FIGS. 3 and 4 are flow diagrams illustrating methods for the preparation of emulsions according to the invention.

### DETAILED DESCRIPTION OF EMBODIMENTS

The principles and operation of emulsions and/or methods of production thereof according to embodiments of the invention may be better understood with reference to the drawings and accompanying descriptions and/or examples.

Before explaining a non-limiting embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

### Materials and Methods

The following exemplary materials are used in experiments described hereinbelow:

Structured surfactant. SLB 365 (Rhodia Inc. USA) was used as a structured surfactant.
Dead Sea water (DSW): Dead Sea Mineral Skin Osmoter™ (Manufacturer Cat. No.: 104030; AHAVA; Dead Sea Laboratories; Israel) was used as the high concentration salt solution. This material has an ionic composition (expressed in mg/L) of Cl (346,000); Mg (92,700); Ca (35,000); Br (14,000); Na (2,720) and K (2,100).
Oil: Sunflower oil (Soetenaey, Fecamp, France) was used as the oil. The refractive index of the oil was 1.330 as measured by refractometer (Atago RX 5000; Japan).
Water: Doubly distilled water (DDW) was used.
Brine: Brine was prepared by dissolving NaCl at a concentration of 10-30 g/L in DDW.
Particle size measurements: Measurements of average particle size in different emulsions were conducted using a Malvern instrument (Nano-S; Malvern Instruments Ltd., Malvern Worcestershire United Kingdom). Briefly, 0.05 gram of an emulsion to be tested were placed in 6 grams of DDW and shaken manually prior to measurement. Each measurement was repeated three times. The following data parameters were used: Dispersant (DDW)—viscosity=0.88718 cps and RI=1.33; Test substrate (sunflower oil)—RI=1.47 and ABS=0. Density was measured by Brookfield DV-I (Stoughton, Mass., USA).

### Example 1

### Exemplary Emulsion Formulations

In order to compare the efficacy of brine versus DSW in stabilizing emulsions, a series of different emulsion formulations were prepared and tested. Table 1 summarizes the various emulsions and their densities.

TABLE 1
Exemplary emulsion formulations of the invention:

| Formulation | Brine % | DSW % | Water % | SS % | Oil phase % |
|---|---|---|---|---|---|
| R | none | none | 62 | 28 | 10 |
| A | none | 10 | 52 | 28 | 10 |
| B | none | 15 | 47 | 28 | 10 |
| C | none | 20 | 42 | 28 | 10 |
| D | none | 25 | 37 | 28 | 10 |
| E | none | 30 | 32 | 28 | 10 |
| F | 10 | none | 52 | 28 | 10 |
| G | 15 | none | 47 | 28 | 10 |
| H | 20 | none | 42 | 28 | 10 |
| I | 25 | none | 37 | 28 | 10 |
| J | 30 | none | 32 | 28 | 10 |

emulsion “R” contained neither DSW nor brine; exemplary emulsions “A” through “E” contained increasing amounts of DSW from 10 to 30% as indicated; control emulsions “F” through “J” contained increasing amounts of Brine from 10 to 30% as indicated; percentages of the structured surfactant (SS) and oil phase were held constant at 28 and 10%, respectively.

As expected, in emulsions A through E of Table 1, with the increase in the percentage of Dead Sea water from 10 to 30%, a corresponding increase in the density of the emulsion from 1.03 to 1.10 was also observed.

### Example 2

### DSW Causes a Persistent Decrease in Particle Size in Emulsions

In order to determine differences between the influence of DSW and the influence of brine on average particle size in emulsion of the invention, the particle size of emulsions A through J of Table 1 was measured at time 0 (immediately after preparation) and then again after a 30-day incubation in a 45-degree centigrade oven (an acceleration model simulating for real stability of shelf life of 12 months).

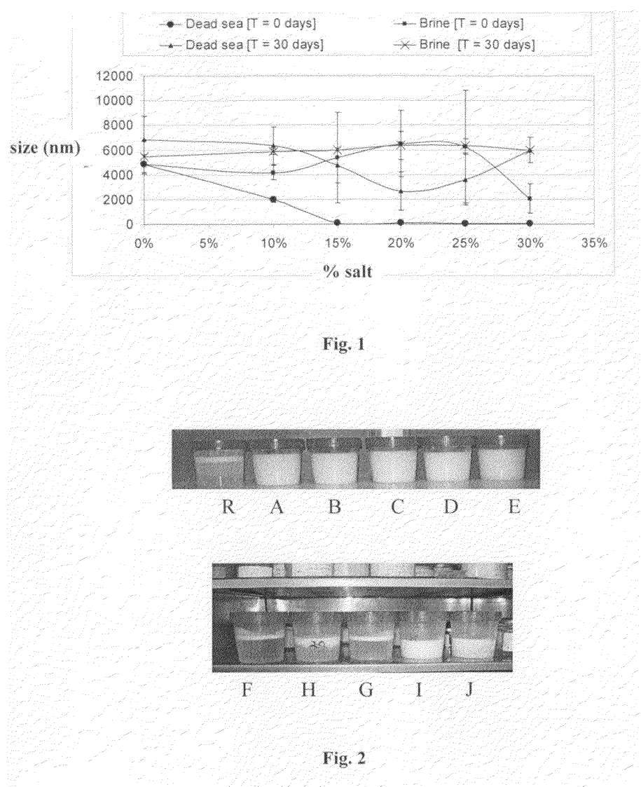

FIG. 1 presents variation in particle size (in nanometers) as a function of the amount of the stabilizing agent (Brine or DSW depending on the emulsion). As FIG. 1 illustrates any incremental reduction in particle size resulting from the increase in brine concentration above 10% is lost after 30 days at 45° C.

In sharp contrast, the incremental reduction in particle size resulting from the increase in DSW concentration above 10% is largely conserved after 30 days at 45° C. Conservation of the reduction in particle size is especially apparent at DSW concentrations of 15 and 20%, decreasing slightly at 25% and again at 30%.

Since 30 days at 45° C. simulates two years of storage at ambient temperature, these results suggest that the use of DSW in place of brine is an effective way to increase average particle size of an emulsion through out a prolonged period of storage.

### Example 3

### Influence of DSW on the Stability of Emulsions

In order to examine the effect of increasing amounts of DSW in emulsions of the invention, jars containing samples of the reference emulsion R and emulsions A through E (Table 1) were placed in a 45° C. oven for two weeks. These conditions simulate storage for 6 months at ambient temperature.

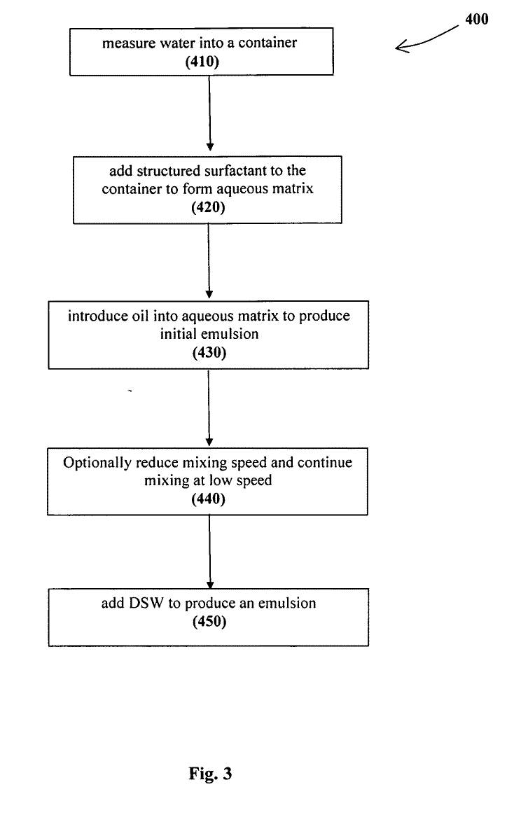

The upper panel of FIG. 2 is a photograph of the jars after simulated storage for 1 year. As may be noted, reference emulsion R has separated into a white oil phase floating on top of a translucent aqueous phase. In sharp contrast, emulsions A through E (emulsions of the invention comprising varying concentrations of DSW) did not separate under identical conditions.

The lower panel of FIG. 2 is a photograph of jars containing emulsions F through J prepared with different concentrations of brine prior to incubation at 45° C. All samples prepared begun separating even prior to the simulated storage.

These results clearly illustrate the unexpected superiority of using DSW to stabilize emulsions. DSW in concentrations of 10 to 30% stabilizes emulsions under storage conditions which would normally bring about phase separation into an oil phase and an aqueous phase. Use of similar concentrations of brine does not stabilize the emulsions in a comparable manner.

### Example 4

### Exemplary Emulsion Preparation Protocol

Emulsions A through J (Table 1) are prepared by incorporating the structured surfactant mixture into DDW and mixing at room temperature (18-25° C.) for about 15 minutes at a low speed (e.g., up to 160 rpm) to form an aqueous phase. Oil (e.g., sunflower oil) was then poured into the aqueous phase during rapid mixing (e.g., 300 rpm) to form an initial emulsion. Optionally, the rapid mixing of the initial emulsion was continued for up to 1 minute after the oil has been added.

The mixing speed of the initial emulsion was then reduced (e.g., to 160 rpm) and mixing was continued for an additional 60 minutes. At this stage, DSW or brine was added to the initial emulsion and stirred for an additional 60 minutes at room temperature at a speed not exceeding 160 rpm.

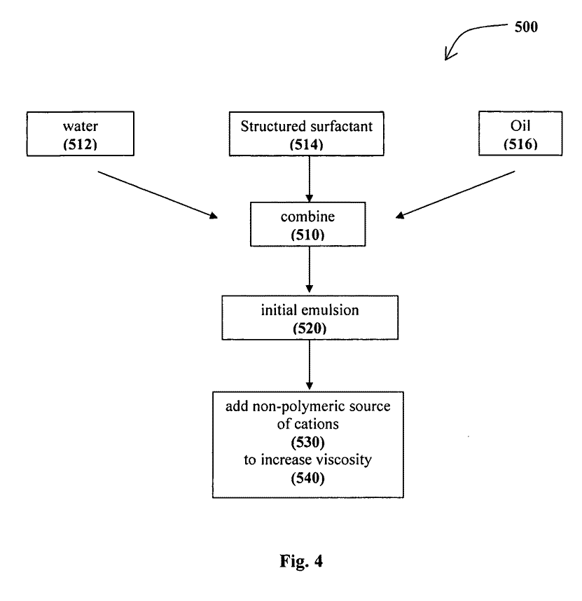

This method is illustrated in FIG. 3, in the form of a flow diagram.

Depicted method 400 includes measuring 410 a quantity of water into a mixing container and adding 420 a quantity of a structured surfactant thereto while mixing at a low speed (e.g., 60 or up to 160 rpm) to form an aqueous matrix. The method 400 further includes introducing 430 an amount of oil into the aqueous matrix while mixing to produce an initial emulsion. The mixing may be at a high speed (e.g., 300 or 400 rpm) and/or for a brief period of time. Further optionally, in the method 400 the mixing speed is reduced 440 to a low speed prior to adding 450 an amount of DSW while mixing to produce a stabilized emulsion.

### Example 5

### Additional Exemplary Emulsion Preparation Protocol

Emulsions A through J (Table 1) were prepared by incorporating the structured surfactant mixture into DDW and mixing in a cup mixer (Visco Jet; Lee; Westbrook Conn.; USA) for about 30 minutes at a low speed (e.g., 60 rpm) to form an aqueous matrix. Oil (e.g., sunflower oil) was then poured slowly into the aqueous phase while increasing the mixing speed (e.g., to 400 rpm) until a uniform initial emulsion was produced (about 15 minutes). At this stage, DSW or brine was added to the initial emulsion and at a low speed and stirred for the same time.

FIG. 4 further provides a flow diagram illustrating an exemplary method of increasing viscosity of an emulsion. Depicted method 500 includes combining 510 an aqueous phase 512 (water), a structured surfactant 514 and oil 516 to produce an initial emulsion 520. Adding 530 a non-polymeric source of cations to the initial emulsion 520 increases 540 the viscosity of the initial emulsion without imparting stickiness to the emulsion.

1-37. (canceled)

38. An emulsion comprising Dead Sea water (DSW).

39. The emulsion according to claim 38, wherein DSW constitutes at least about 10% w/v of the emulsion.

40. The emulsion according to claim 38, further comprising at least one structured surfactant.

41. The emulsion according to claim 38, further comprising water; at least one oil; DSW; at least one structured surfactant; and optionally at least one additive.

42. The emulsion according to claim 41, wherein the DSW constitutes at least about 10% w/v of the emulsion.

43. The emulsion according to claim 41, wherein the DSW constitutes at most 30% w/v of the emulsion.

44. The emulsion according to claim 38, wherein the DSW is natural DSW.

45. The emulsion according to claim 38, wherein the DSW is a salt solution simulating natural DSW.

46. The emulsion according to claim 41, wherein the at least one structured surfactant constitutes at least about 20% w/v of the emulsion.

47. The emulsion according to claim 41, wherein the at least one structured surfactant constitutes at most about 40% w/v of the emulsion.

48. The emulsion according to claim 41, wherein the at least one structured surfactant is a combination of at least one surfactant selected from the group consisting of at least one anionic surfactant, at least one nonionic surfactant, at least one amphoteric surfactant, at least one zwitterionic surfactant and at least one cationic surfactant, with at least one agent selected from the group consisting of water, at least one anionic surfactant, at least one electrolyte, and at least one alkanolamide.

49. The emulsion according to claim 41, wherein the oil constitutes at least about 3% w/v of the emulsion.

50. The emulsion according to claim 41, wherein the oil constitutes at most about 30% w/v of the emulsion.

51. The emulsion according to claim 41, wherein the oil comprises at least one oil selected from the group consisting of a vegetable oil, an essential oil, botanical oil, a mineral oil and an animal oil.

52. The emulsion according to claim 51, wherein the oil comprises at least one vegetable oil.

53. The emulsion according to claim 38, formulated as a personal skin care product.

54. The emulsion according to claim 53, wherein the personal skin care product is selected from the group consisting of a cleansing product and a moisturizing product.

55. A dermatological formulation comprising the emulsions of claim 38.

56. A method for treating a skin disease or disorder in a subject, comprising topically applying a therapeutically effective amount of the dermatological formulation of claim 55 to the skin of the subject in need thereof.

57. A formulation comprising at least one emulsion according to claim 38.

58. A method of producing a non-sticky emulsion, comprising: combining water, at least one structured surfactant and at least one oil to produce an initial emulsion; and adding a non-polymeric source of cations to the initial emulsion, to thereby increase the viscosity of the emulsion without imparting stickiness to the emulsion.
